(19) United States
(12) Patent Application Publication
(10) Pub. No.: US 2009/0170091 A1
(43) Pub. Date: Jul. 2, 2009

(54) Method For Predicting Biological Systems Responses

(76) Inventors: Kenneth Giuliano, Pittsburgh, PA (US); Albert H. Gough, Glenshaw, PA (US); Patricia A. Johnston, Sewickly, PA (US); D. Lansing Taylor, Pittsburgh, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/087,809

(22) PCT Filed: Jan. 17, 2007
(86) PCT No.: PCT/US2007/001217

§ 371 Date: Feb. 6, 2009

Related U.S. Application Data
(60) Provisional application No. 60/759,476, filed on Jan. 17, 2006.
(60) Provisional application No. 60/846,006, filed on Sep. 20, 2006.

(51) Int. Cl. C12Q 1/68

(52) U.S. Cl. 435/6

(57) ABSTRACT

The inventive method employs a “systems biology” approach to predicting biological responses resulting from exposure to the test substance. In one embodiment, the invention provides an automated method for predicting the biological systems effects of a test substance. In another embodiment, the invention provides a method for constructing a knowledgebase (or database) of response profiles for reference substances with known biological systems effects. In another embodiment, the invention provides a set of protocols and software tools used to carry out the profiling. Another embodiment of the invention is a panel of reagents and protocols required for generating response profiles, either to create an knowledgebase, or to use with an existing knowledgebase and informatics software to profile substance physiological effects. Another embodiment of the invention is a database of physiological profiles.

### CROSS-REFERENCE TO RELATED APPLICATIONS

This patent application claims the benefit of U.S. Provisional Patent Application No. 60/759,476, filed Jan. 17, 2006, and U.S. Provisional Patent Application No. 60/846,006, filed Sep. 20, 2006. The entire contents of these provisional patent applications are incorporated herein in their entireties.

### BACKGROUND OF THE INVENTION

Assays aimed at predicting biological responses to test substances are central to activities such as drug discovery, personalized medicine, environmental toxicology and biomedical research. Typically, assays are conducted to assess the effect of a test substance on a predefined target, which could be molecular or cellular behavior. In the area of basic biological research and medical research, for example, cell analysis is routinely used. Some such research is directed at drug discovery, and such research can identify potential drug candidates, which undergo extensive series of preclinical and clinical studies. Yet, many candidate drugs fail because safety (e.g., toxicity) and/or efficacy concerns are discovered only in late stage clinical trials in humans. This results in inefficiency that could be reduced by the use of earlier-stage assays predictive of the action of a drug candidate in vivo.

Personalized medicine is an emerging discipline that is based on a systems approach to disease that takes into account a profile of the whole patient, to determine the most effective therapy. The molecular information derived from genomics and proteomics, and in particular those genes and proteins that have been correlated with particular disease conditions (often referred to as “biomarkers”), is certainly a valuable source of patient data. However, customization of medical treatment through this approach is limited to well characterized classes of biomarkers, since therapies cannot be tested for every individual genome without improved methods of cellular analysis.

The challenge in environmental toxicology is to assess the impact of a growing list of substances on human health. Several factors complicate the problem, such as increasingly large numbers of substances to be tested; the complexities of environmental exposure require testing over a broad range of exposure mechanism, concentration and time; and uncertainties regarding the influence of age and genetic variability on the results. Reliable means to improve the efficiency of environmental toxicology testing, and to reduce the number of animal tests required, are actively being sought by the National Toxicology Program at the United States National Institutes of Health and other governmental and private sector entities worldwide

In these areas, and others in which cellular assays are central, progress is limited by assays that are typically focused on a single cellular process, as there are limited tools available for analyzing complex, multi-component system responses. A recent comparison of the performance of a panel of cytotoxicity assays, including DNA synthesis, protein synthesis, glutathione depletion, superoxide induction, Caspase-3 induction, membrane integrity and cell viability found that these assays on average had only half the predictive power of animal studies (Xu et al., Chem Biol Interact, 2004. 150(1): p. 115-28.). However, these assays were carried out independently, and no attempt was made to combine the readouts in any quantitative way, to improve the overall predictivity. Several studies have shown that the multidimensional cellular responses from cell-based assays can be clustered using standard methods, to identify compounds with similar activities (Taylor et al., Drug Discov Today, 2005. 2(2): p. 149-154; Mitchison, Chembiochem, 2005. 6(1): p. 33-9; Perlman, Science, 2004. 306(5699): p. 1194-8). These studies have demonstrated proof of principle for clustering compound responses, but have not attempted to correlate these identified clusters with specific response profiles and then use the response to predict the physiological impact of unknown substances. A simple automated classifier has been developed for use with some commercially available assays. This classifier allows the use of Boolean operations to combine the outputs from several assay features into a single result (Abraham et al., Preclinica, 2004. 2(5): p. 349-355). These Boolean operations allow the assay developer to define an output that combines several feature measurements. This is very useful in expanding the scope of some high content screening (HCS) assays, but has limited features, and is certainly not designed for, nor would it be easy to use with multidimensional feature sets. Accordingly, there is a need for a more robust method for predicting biological systems responses.

### BRIEF SUMMARY OF THE INVENTION

In one embodiment, the invention provides an automated method for predicting the biological systems effects of a test substance. In accordance with one aspect, a battery of cells to be treated with the test substance is provided, and the cells to be treated contain a unique combination of fluorescent or luminescent reporters or manipulations. The reporters respond to and indicate a functional response, whereas the manipulations produce a functional response in the cells. Either before or after addition of the reporters or performing the manipulations, the cells are contacted with (incubated with) the test substance. After the addition of the reporters or performing the manipulations and contacting the cells with the test substance, cells are imaged or scanned to obtain fluorescence images of the reporters. Thereafter, images of the cells are analyzed to measure or detect cellular features. Thereafter, these features from the cells are combined to produce a response profile for the test substance. In accordance with another aspect, a battery of cells to be treated is provided, which is similarly incubated with the test substance. Thereafter, images of cells within the battery are acquired and analyzed to measure or detect cellular features indicative of cellular functional classes. Thereafter, these features from the cells are combined to produce a response profile for the test substance. In either aspect, the method involves finally comparing the response profile of the test substance to a database (or knowledgebase) of response profiles for reference substances with known biological systems effects. As a result of such comparison, the extent of correlation between the response profile of the test substance to the database of response profiles for substances with known biological systems effects indicates the probability that the test substance will exhibit a biological systems effect in a living cell, tissue or organism.

In another embodiment, the invention provides a method for constructing a knowledgebase (or database) of response profiles for reference substances with known biological systems effects. In accordance with one aspect, a battery of cells to be treated with the test substance is provided, and the cells to be treated contain a unique combination of fluorescent or luminescent reporters or manipulations. Either before or after addition of the reporters or performing the manipulations, the cells are contacted with (incubated with) a reference substance. After the addition of the reporters or performing the manipulations and contacting the cells with the reference substance, cells are imaged or scanned to obtain fluorescence images of the reporters. Thereafter, images of the cells are analyzed to measure or detect cellular features. Thereafter, these features from the cells are combined to produce a response profile for the reference substance. In accordance with another aspect, a battery of cells to be treated is provided, which is similarly incubated with the reference substance. Thereafter, images of cells within the battery are acquired and analyzed to measure or detect cellular features indicative of cellular functional classes. Thereafter, these features from the cells are combined to produce a response profile for the test substance. In either aspect, the method involves comparing the response profile of the test substance to a database (or knowledgebase) of response profiles for reference substances with known biological systems effects. The response profile for the reference substance then is added to the database. The steps can be repeated using different reference substances (e.g., first reference substance, second reference substance, etc.) to increase the database. The invention also provides a knowledgebase (or database) of response profiles.

The method can result in the identification and classification of predicted in vivo functional responses for applications in drug discovery, personalized medicine, environmental toxicology, biomedical research and in other fields (e.g., environmental health and industrial safety).

In another embodiment, the invention provides a set of protocols and software tools used to carry out the profiling. Another embodiment of the invention is a panel of reagents and protocols for generating response profiles, either to create a knowledgebase (or database), or to use with an existing knowledgebase (or database) and informatics software to profile substance physiological effects. Another embodiment of the invention is a database or knowledgebase of physiological profiles.

These aspects, and other inventive features, will be apparent from the accompanying drawings and following detailed description.

### BRIEF DESCRIPTION OF THE SEVERAL VIEWS OF THE DRAWING(S)

FIGS. 1A and 1B presents a flowchart of one embodiment of the inventive method. FIG. 1A concerns construction of the database or knowledgebase and FOG/ 1B concerns assessing a test compound using the database or knowledgebase.

FIG. 2 depicts exemplary images from multiplexed HCS assays.

FIG. 3 illustrates the sample flow while processing plates to produce profiles in accordance with the inventive method.

FIG. 4 illustrates some graphical display methods to display cellular responses that contribute to creating a cellular response profile.

FIG. 5 illustrates some graphical display methods to display cellular responses that contribute to creating a cellular response profile. FIG. 5a depicts an array of cellular response data plots. 5b depicts a three-dimensional surface plot. FIG. 5c depicts a two-dimensional contour plot or “Distribution Map” of the data.

FIG. 6 illustrates a combination of six toxicity-related functional classes associated with toxicity assessment and corresponding cellular features.

FIG. 7 illustrates standard plate layouts for CellCipher Cytotox Profiling Multiplex Plates 1 and 2 described in Example 6.

FIG. 8 presents data generated by the CellCipher analysis based on dose response described in Example 6.

FIG. 9 presents data demonstrating CellCipher profile clustering analysis of a 30 compound test set as described in Example 6.

FIG. 10 demonstrates CellCipher profile principal component analysis of a 30 compound test set.

### DETAILED DESCRIPTION OF THE INVENTION

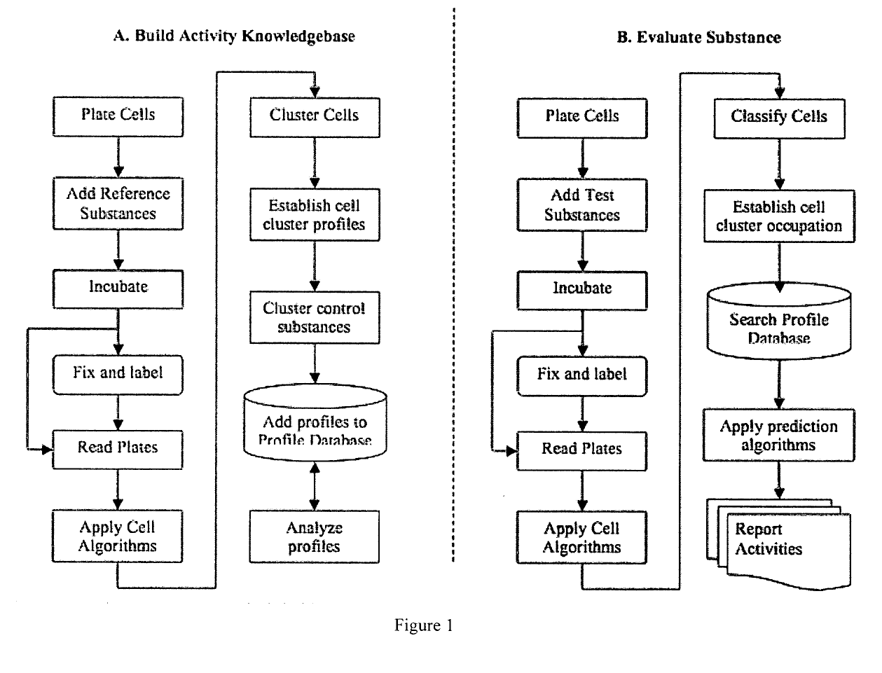

The inventive method employs a “systems biology” approach to predicting biological responses resulting from exposure to the test substance. The method is based on integrating cell-based assays of multiple components of a cell system to generate response profiles that are predictive of higher level cell and cell system and organism functions and responses. Embodiments of the inventive method are presented in a flow chart in FIGS. 1A and 1B. For building such a database or knowledgebase, the response profile of a reference substance is determined and added to the database (Figure IA). For assessing a test substance, the response profile of the test substance is compared to a database (or knowledgebase) of response profiles for reference substances with known biological systems effects (FIG. 1B).

The inventive method is conducted using a battery of cells to be treated with the test or reference substance. The cells within the battery to be tested can be from a single cell type or multiple cell types. The use of multiple cell types can, however, more broadly indicate tissue associated responses. Cell types typically are selected based on the target function of the assay. For example, for toxicity profiling, hepatocytes, cardiomyocytes, or microvascular endothelial cells can be selected. Such cells can be primary cultures or established cell lines (e.g., HepG2), as desired, and are commercially available from a variety of sources (e.g., Amphioxus, Admet Technologies, Multicell Technologies, Cambrex (Clonetics), Cellular Dynamics, CXR Bioscience, Cambrex, Cell Applications, Inc., and Geron (Cxr Bioscience)). The cells within the battery can be of one type or a mixture of cell types, as desired.

The cells within this battery can optionally contain one or more reporters and/or manipulations. In some embodiments, each cell within the battery of cells contains a unique combination of reporters and/or manipulations. In other embodiments, populations of cells within the battery contain unique combinations of reporters and/or manipulations. The cells should contain a number of reporters and/or manipulations suitable to approximate a biological system. Typically, the cells contain a unique combination of at least 6 or more (such as at least about 7 or more, or at least about 8 or more) and even at least about 10 or more or at least about 15 or more unique combinations of reporters and/or manipulations.

In the context of the inventive method, a “reporter” is a fluorescent or luminescent molecule, such as a physiological indicator, label, a protein, a biosensor, etc. The reporter can be a protein or non-proteinaceous. Where a reporter is proteinaceous, however, the cells can express one or more of the reporter molecules. Alternatively or additionally, one or more of the reporter molecules can be delivered into the cell, e.g., by attaching a protein sequence tag facilitating importation across the plasma membrane. In embodiments where the cells are fixed prior to imaging, a reporter can be provided by standard labeling technology.

Examples of labels that are suitable reporters for use in the context of the inventive method include, for example, probes available to label subcompartments, localize proteins, label membranes, respond to membrane potentials, sense the local chemical environment, read out molecular mobility, and provide many other measurements (see, e.g., Waggoner, A., “Fluorescence probes for analysis of cell structure, function and health by flow and imaging cytometry.,” in Applications of Fluorescence in the Biomedical Sciences, D. Taylor, et al., Editors. 1986, Alan R. Liss, Inc.: New York. p. 3-28.). Coupled with antibodies, immunofluorescence labeling provides an easy method for detecting and localizing proteins or protein variants such as phosphorylated proteins. Cells also can be engineered to express proteins tagged with any of the color variants of fluorescent proteins (Chalfie et al., Science, 1994. 263(5148): p. 802-5; Chudakov, et al. Trends Biotechnol, 2005. 23(12): p. 605-13), and these fluorescent proteins can be further engineered to create biosensors, indicators of specific cellular functions (see, e.g., Conway et al., Receptors Channels, 2002. 8(5-6): p. 331-41; Umezawa, et al., Biosens Bioelectron, 2005. 20(12): p. 2504-11; Giuliano et al., Trends Biotechnol, 1998. 16(3): p. 135-40; Giuliano et al., Curr Opin Cell Biol, 1995. 7(1): p. 4-12). A variety of labels can be combined in a single sample preparation to provide for the measurement of many features in each individual cell in a population, as well as in the population as a whole (Zhang et al., Cell, 2004. 119(1): p. 137-44; Taylor et al., Drug Discov Today, 2005. 2(2): p. 149-154). Quantum dots, with their single excitation wavelength and narrow emission bands, provide the potential for even higher degrees of multiplexing within an assay (Michalet, et al., Science, 2005. 307(5709): p. 538-44). In addition the rainbow of fluorescent probes, a number of bioluminescent and chemiluminescent reagents can be effectively used in cell based assays (Hemmila et al., J Fluoresc, 2005. 15(4): p. 529-42; Roda et al., Trends Biotechnol, 2004. 22(6): p. 295-303).

In the context of the inventive method, a “manipulation” is a treatment of one or more cells to effect a functional response (or change) in the cell. Cells can be manipulated using chemical, biological, environmental, or genetic treatments. These treatments can be used to alter the activity of cellular ions, metabolites, macromolecules, and organelles, which, in turn, effect phenotypic changes that can be further altered by treatment with additional substances. Examples of manipulations include expression or heightened expression of a protein, knock-down of the expression of a protein, addition of a stimulus of known response or addition of a substance which induces differentiation of stem cells or precursor cells. In one embodiment, intracellular ion concentrations can be altered (manipulated) by treating cells with ionophores such as ionomycin to modulate intracellular free calcium ion concentration or cells are treated with nigericin to modulate intracellular pH. In another embodiment, cells can be treated with substances to manipulate the concentration of intracellular metabolites. For example, treatment of cells with forskolin, 8-Br-cAMP, or dibutyryl-cAMP alters the intracellular concentration of the signaling metabolite cAMP. In another embodiment, cells can be manipulated to alter the activity and concentration of intracellular macromolecules. For example, macromolecules such as proteins can be introduced into cells using physical perturbation methods such as microinjection or cell scraping. Alternatively, the normal expression levels of proteins in cells are decreased by introducing molecules such as siRNAs, miRNAs, or antisense-RNAs into cells. In this sense, for instance, Cdc2 siRNA pretreatment can be employed to induce a G2 cell cycle block in the cells, which can be employed for assaying the test compound for inhibition of apoptosis-inducing activity. As another example, the normal expression levels of macromolecules in cells can be increased using inducible expression systems such as those employing insect-based (e.g., ecdysone) or antibiotic-based (e.g., tetracycline) molecules to control the expression of genes encoding proteins as well as RNA molecules that encode either proteins or other macromolecules. Furthermore, RNA molecules can be introduced into cells that modulate the level or activity of other non-coding RNAs such as miRNAs, RNAs transcribed as part of protein introns, and any other primary or secondary RNA molecules that arise from transcription of any part of the genome or any other genetic material within the cell.

The cells are plated on substrates such as microplates, microscope slides or other labware typically used for cell based assays. Generally, such labware is transparent to facilitate subsequent imaging analysis. Multiwell microplates are preferred as they facilitate multiple iterative assays to be conducted simultaneously and can be readily handled using automated equipment. The cells can be plated at any desired density to facilitate subsequent imaging analysis. For multiwell microplates, several thousand cells can be introduced into each well (e.g., 7000-8000 cells per 40 μl well).

Once plated, the cells are contacted with a test substance or a reference substance. In the context of the present invention, the “test substance” or “reference substance” is any substance, the response profile of which within a complex cell system or organism is desired. For example, a test or reference substance can be a small molecule (such as a “drug” or drug candidate), a biomolecule (such as a protein, polypeptide, nucleic acid (e.g., DNA, RNA, or hybrid polynucleotides)), an environmental condition (such as osmolality, pH, temperature or a combination thereof), electromagnetic radiation (e.g., light frequency, intensity, or duration), or other types of radiation (e.g., alpha, beta, gamma radiation, etc.). A substance is treated as a test substance when its effect on the biological system in question is being probed. A substance is a reference substance when its effect on the biological system is known and where its effect on the battery of cells is desired to that its profile can be added to the database or knowledgebase.

In performing the inventive method, a test or reference substance is exposed to the cells in a manner suitable for the test or reference substance to come into contact with the cells and interact with the cells. Typically, where the test or reference substance is a molecule, it can be introduced into the location of the cells (e.g., a well of a culture plate into which the cells are placed). The molecule then can interact with the cell at its outer surface or permeate the cell and interact with its internal workings. Other types of test or reference substances (e.g., temperature, radiation, etc.) are exposed to the cells in a manner suitable to the type of substance. The cells are incubated with the test or reference substance for a suitable time, which can vary from one or a few minutes to several days. The length of time can be selected based on whether immediate or chronic activity is desired, for example.

In alternative embodiments, iterative batteries of cells (i.e., similar batteries) can be treated in parallel employing differing test substance or reference substance concentrations so that a response profile can be constructed for each concentration. For example, 6-10 point log concentration series can be employed for compounds ranging in concentrations from about 1 nM or less to about 1 mM or greater. Similarly, different batteries of cells (e.g., having a different set of reporters or manipulations) can be exposed to the test substance. Employing iterative batteries of either different cell type and/or concentration can thus be conducted in parallel (e.g., in different wells of the same multi-well plate) and analyzed concurrently or in parallel. Also, negative and positive control cells (e.g., untreated wells or wells treated with a substance with a known activity) can be assayed along with the test substance or reference substance(s).

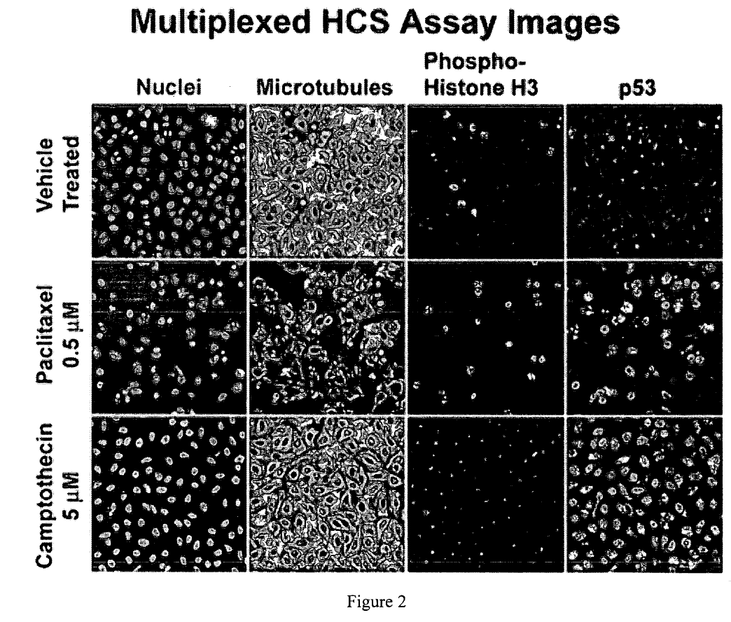

After the test or reference substance is exposed to the cells, images of the cells are acquired. Where the cells contain one or more reporters, images are obtained using frequencies (channels) appropriate for each of the fluorescent or luminescent reporters to be imaged. An example of such multiplex images is presented in FIG. 2. Additionally or alternatively, the cells can be stained with dyes, fluorescent or luminescent labels (e.g., antibodies, ligands, etc.) that bind to desired proteins or cellular structures, and then imaged at frequencies (channels) appropriate for each of the dyes, fluorescent or luminescent labels to be imaged.

The images of the cells are analyzed to measure or detect cellular features, which are selected to be indicative of the functional classes appropriate to the property (such as toxicity, clinical pathology, histopathology, etc.) to be assayed. Thus, the reporters (labels, dyes, etc.) can be selected to target (e.g., bind to) features appropriate for assaying classes of cellular function. Within each of these cellular function classes, one or more assays are used to measure one or more of the cellular features as an indication of a response in that assay function class. In some embodiments, a single reporter corresponds to a single feature. In other embodiments, a reporter can be used to assess different features.

Any suitable cellular functional classes can be selected, depending on the aim of the assay. Examples of cellular features and function classes suitable for assessing toxicity are presented in Example 1. In a preferred embodiment, the cellular features are selected from 2 or more functional response classes in the group consisting of cell proliferation, stress pathways, organelle function, cell cycle state, morphology, apoptosis, DNA damage, metabolism, signal transduction, cell differentiation and cell-cell interaction. In another preferred embodiment, the cellular features are selected from 2 or more functional response classes in the group consisting of cell proliferation, cell cycle, apoptosis, oxidative stress, stress kinase activation, mitochondrial function, DNA damage, and peroxisome proliferation. Cellular features indicating cell proliferation that can be assayed include nuclear count, cell count, total cell mass, total DNA, the phosphorylation state of cell cycle regulatory proteins, or the post-translational modification state of any protein involved in cell growth or division. Furthermore, cellular features indicating stress pathway activation that can be assayed include transcription factor activation of NF-κB, P1, ATF2, MSK1, CREB, or NFAT, or kinase activation of p38, JNK, ERK, RSK90 or MEK. Furthermore, cellular features indicating organelle function that can be assayed include cytoskeletal organization, mitochondrial mass or membrane potential, peroxisome mass, golgi organization, or plasma membrane permeability. Furthermore, cellular features indicating cell cycle state that can be assayed include DNA content, Histone H3 phosphorylation state, Rb phosporylation state, cyclin B1 (CDK1) biosynthesis, cyclin D1 (CDK4, 6) biosynthesis, cyclin E (CDK2) biosynthesis. Furthermore, cellular features indicating morphology that can be assayed include motility, cell spreading, adhesion, ruffling, neurite outgrowth or colony formation. Furthermore, cellular features indicating apoptosis that can be assayed include nuclear size and shape, DNA content and degradation, caspase activation, phosphatidyl-expression, Bax translocation. Furthermore, cellular features indicating DNA damage that can be assayed include repair protein (APE) expression, tumor suppressor (p53, Rb) expression. Oxidative activity (8-oxoguanine), or transcription activity (Oct1). Furthermore, cellular features indicating metabolism that can be assayed include cAMP concentration, P-glycoprotein activity or CYP450 induction/inhibition, or the concentration of an added substance. Furthermore, cellular features indicating signal transduction that can be assayed include Ca++ ion concentration, pH, expression of a protein, activation of a protein, modification of a protein, translocation of a protein, or interaction between proteins known to be associated with a specific pathway. Furthermore, cellular features indicating cell differentiation that can be assayed include a tissue specific protein or exhibiting a tissue specific morphology. Furthermore, cellular features indicating cell-cell interactions that can be assayed include concentration of tight junction proteins at a cell-cell interface, or transfer of material from one cell to another. Preferred cellular features that can be assayed include microtubule stability, histone H3 phosphorylation, mitochondrial mass, mitochondrial membrane potential, p53 activation, c-jun phosphorylation level, histone H2A.X phosphorylation level, nuclear size, cell cycle arrest, DNA degradation, and cell loss.

The imaging to assay the desired cellular features can be conducted using fixed or live cells. For live cell assays, labeling reagents (reporters) are optionally added before the plate (or other substrate) is scanned or read. Fixation and labeling (or staining) with reporters such as antibodies, dyes, etc. is routine and can be automated, allowing efficient processing of assays. For fixed cell assays, spatial information is acquired, but only at one time point. However, where iterative assays are conducted in parallel, it is possible to fix cells in separate wells at desired time intervals (e.g., every second, every minute, etc.) to facilitate analysis of like populations of cells over time. By contrast, live cell assays permit an array of living cells containing the desired to be imaged over time, as well as space. However, environmental control of the cells (e.g., temperature, humidity, and carbon dioxide) is required during measurement, since the physiological health of the cells must be maintained for multiple luminescence or fluorescence measurements over time. For either live or fixed cell assays, scanning of the cells (or of separate subpopulations of the cells) can be repeated multiple times to facilitate analysis at each time point to capture a kinetic response to the test or reference substance.

Acquiring images of the cells and analysis to extract cellular features can be accomplished by standard methods and equipment (e.g., Schroeder et al., J. Biomol. Screen, I(2), 75-80 (1996); Taylor et al., Toxicol. Pathol., 22(2), 145-59 (1994)), such as High Content Screening (HCS) (e.g., Giuliano et al., J Biomol Screen, 1997. 2(4): p. 249-259) and high throughput cell analysis, automated microscope, or other detector. Briefly, the instrument is used to scan one or more optical fields in each sample or microplate well, collecting one or more channels of fluorescence for each optical field. The multiwavelength images allow a panel of assays to be multiplexed in a single preparation, but assays can also be run across multiple preparations, and the feature measurements combined into a single activity profile. The extraction of cellular features can be accomplished during image acquisition, or the images can be acquired and processed later. Suitable instruments include those for analysis of cell population responses on a whole plate at once, such as the FLIPR (Molecular Devices, Sunnyvale, Calif.) or FDSS 6000 (Hamamatsu City, Japan), as well as instrumentation for well-by-well and cell-by-cell analysis, such as the ArrayScan® HCS reader (Cellomics, Pittsburgh, Pa.); fixed endpoint and kinetic cell-based assays; image analysis algorithms that generate the primary cell response data; and data analysis tools for extracting derived features such as kinetic parameters, EC₅₀, IC₅₀, and population response distributions from the measurements. The assays can include combinations of HCS assays where individual cells are measured, along with higher throughput assays where the population of cells in a well is analyzed as a whole, either at a single time point, or at multiple time points to measure a kinetic response. For kinetic assays, multiple features can be extracted from the kinetic curve to create additional derived features. For example, features such as delay to peak, peak intensity, half time of decay, slope, and others can be derived from kinetic curves.

An algorithm is used to extract information from the images to produce outputs of different cellular features. Typically, such algorithms convert raw image data to assay data points. Those skilled in the art of imaging and cell analysis will recognize that many such algorithms are readily available, and that there are many such cellular processes that are amenable to image-based analysis of cells to measure cellular functions. The algorithms, custom designed or encapsulated in the BioApplication software provided by the HCS vendors, produce multiple numerical feature values such as subcellular object intensities, shapes, and location for each cell within an optical field. The vHCS™ Discovery Toolbox (Cellomics, Inc), Metamorph™ (Molecular Devices), software from GE Healthcare and other HCS and image analysis packages can be used to batch analyze images following acquisition. In such systems, the total number of cells measured per well is typically in the range of 100-1500, depending on the heterogeneity of the cellular response and the sensitivity of the assay. Whole plate readers are typically supplied with software to identify well areas in the image and measure the total fluorescence in those areas for one or more time points.

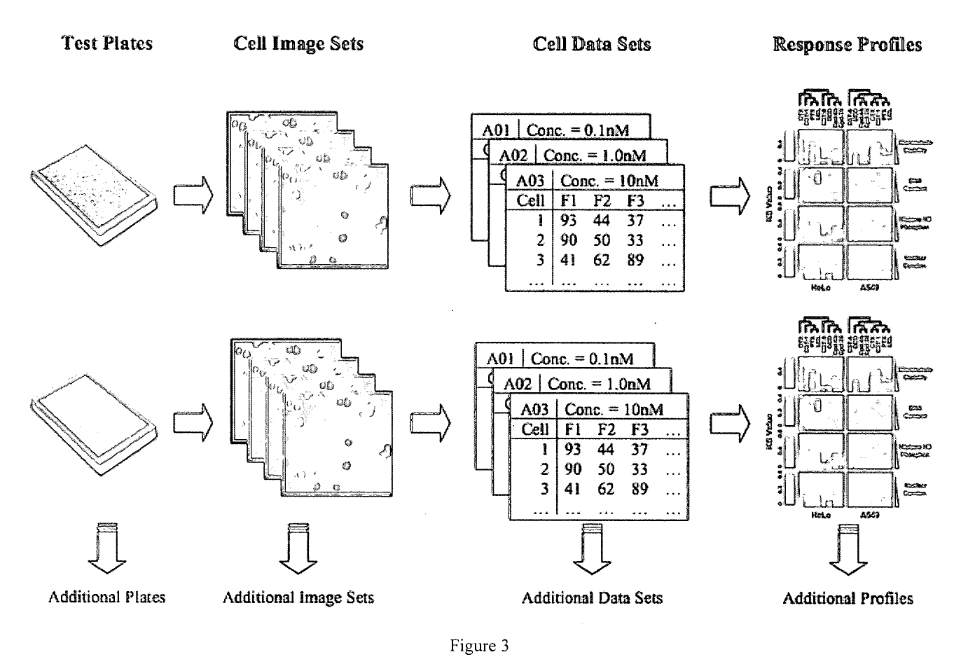

Desirably, an algorithm is used to combine outputs of different cellular features and assays from one or more or more assay plates or wells to produce a compound response profile suitable for predicting higher level integrated functions. Features can be combined for cells or plates at different time points (e.g., where a physiological response occurs over a period of time). Alternatively, iterative experiments using different cell types in different wells or plates can be similarly combined. Preferably, the response profile represents at least 6 or more features or functional classes (such as at least about 7 or more, or at least about 8 or more) and even at least about 10 or more or at least about 15 or more features or functional classes. Each plate in the plate set can produce an image set consisting of images from one or more fields in each well, at each of the wavelengths and time points to be analyzed. Analysis of the image set produces a cell data set for each plate representing feature values over time and over concentration series for each field imaged on the plate. Finally, the cell data sets are processed and clustered to produce a set of response profiles to be added to the database or knowledgebase, or to be used to search the database or knowledgebase to identify probable modes of physiological response. FIG. 3 illustrates the overall sample flow while processing plates to produce profiles.

Several methods can be used to generate the profiles from the feature measurements. For example, a parameter such as Kolmogorov-Smirnov (KS) values or average values as a measure of cell population shifts can be calculated for each feature measurement at each compound concentration for each compound, which results in the generation of parameters dilution series. Such dilution series parameters then can be fitted, using a 4-parameter logistic fit and the resulting fitted data analyzed to calculate EC₅₀ values. The calculated EC₅₀ values can, in turn, be converted to a log scale as a measure of test substance or reference substance activity. Cluster analysis then can be used to identify similarities in profiles as well as correlations between cellular systems responses.

FIG. 1A illustrates one embodiment for producing the reference response profiles to construct the database or knowledgebase. In accordance with this method, some assay data points generated by the algorithm can be analyzed to identify 2 or more subpopulation of cells. For example, the intensity of nuclear labeling is related to the amount of DNA in the nucleus. The nuclear intensity data from a population of cells in a well can be analyzed to identify cells with 2N, 4N and sub 2N amounts of DNA, the latter being an indication of DNA breakdown. The population of cells can thus be clustered into subpopulations based on 1 or more assay values, each subpopulation having a characteristic profile of those assay values, and therefore representing a class of cellular response. In this example there are three subpopulations and therefore three features consisting of the percentage of cells with 2N DNA, the percentage of cells with 4N DNA, the percentage of cells with sub 2N DNA. Each of these features can be usefully included as a component of the compound profile. Combinations of any number of other assay features can also be used to classify cells into subpopulations. Some assay features, such as fraction cell loss, are characteristics of the whole population and therefore are used directly as a component in the compound profile. In all cases assay values for treated cells are compared with cells treated with vehicle (e.g. 0.4% DMSO) alone.

Compound profiles are subjected to cluster analysis, principle component analysis and other pattern analysis methods to identify common response profiles among a collection of compounds. These clusters of compounds represent a common class of response, and the profile of that response can be used to construct a classifier. The profiles of all the reference compounds along with the profiles of compound classes are stored in a Profile Database for additional pattern analysis.

FIG. 1B illustrates one embodiment for producing the response profiles involving evaluating a test compound, and classification of the compound response. As with the analysis of the reference compounds, the assay features are further analyzed to identify cell subpopulation profiles which along with the direct assay features form the compound profiles which are stored in the database. A measure of the similarity between the response profile of a test compound and the response profile in the database or knowledgebase is used to calculate a probability that a test compound would produce the associated profile in vitro or in vivo. The metric used to compare compound profiles can be any of a number of standard metrics such as Euclidean distance, Pearson's correlation coefficient, Manhatten distance, or any other metric for comparing multiparameter profiles. Test compounds profiles are analyzed with reference compounds to identify linkage of the test compound with a particular cluster. Those skilled in the art will recognize that there are a variety of linkage models as well as other classification approaches that can be used to classify test compounds relative to reference compound profiles in the database.

In another embodiment of the invention, all the cell feature values from each cell are combined to create a cell profile. The cell profiles from the populations of cells treated with reference compounds are clustered, to identify specific response classes. All the cells in a single well, and therefore exposed to a particular substance at a specific concentration, are classified into these response classes. The percent occupation of each of these classes then becomes a population response profile for that well. The population profiles from the reference compounds are linked to the profiles from the reference compounds and stored in the database or knowledgebase. The population profiles from the test compounds are compared with the population profiles of the reference compounds in the database and the probability of a match is calculated.

To quantify changes in the cellular responses induced in a population of cells by treatment with reference or test substances, several different methods can be effectively used. Within a population of cells, many different individual cellular response profiles are possible, due to the heterogeneity in cellular responses (Elsasser, Proc Natl Acad Sci USA 1984; 81 (16):5126-9; Rubin H, Proc Natl Acad Sci USA 1984; 81 (16):5121-5). In one embodiment, the cellular response distribution for each cell parameter in a well or on a slide is compared with that of a control substance using a Kolmogorov-Smirnov (KS) goodness of fit analysis (KS value) (Giuliano et al., Assay Drug Dev Technol 2005; 3 (5):501-14). To perform significance testing of substance dependent changes in multiplexed HCS-derived cell population distribution data, the one-dimensional KS test can be adapted to two dimensions as described by Peacock (Peacock, Monthly Notices of the Royal Astronomical Society 1983; 202:615-27) and further refined by Fasano and Franceschini (Fasano et al., Monthly Notices of the Royal Astronomical Society 1987; 225:155-70.). The two-dimensional cell population data distributions representing two physiological parameters from a multiplexed HCS assay obtained after treatment with a substance can be compared to the two-dimensional cell population data distributions obtained from multiple wells of untreated cells. First, each distribution can be divided into quadrants defined by the median x and y axis values calculated from the untreated cell data distributions. The two-dimensional KS value can then be found by ranging through all four quadrants to find the maximal difference between the fraction of cells in each treated quadrant and the fraction of cells in each corresponding untreated quadrant. The heterogeneity of cell population responses can also be analyzed with other statistical methods. Several other possible analysis algorithms or methods can be used to classify cell response profiles based on the known properties of a training set of reference substances, including methods such as neural nets. KS response profiles can be clustered by agglomerative clustering, to identify compounds with similar activities. Other methods in addition to KS analysis can be used to process data prior to clustering, and a variety of clustering algorithms can be usefully applied.

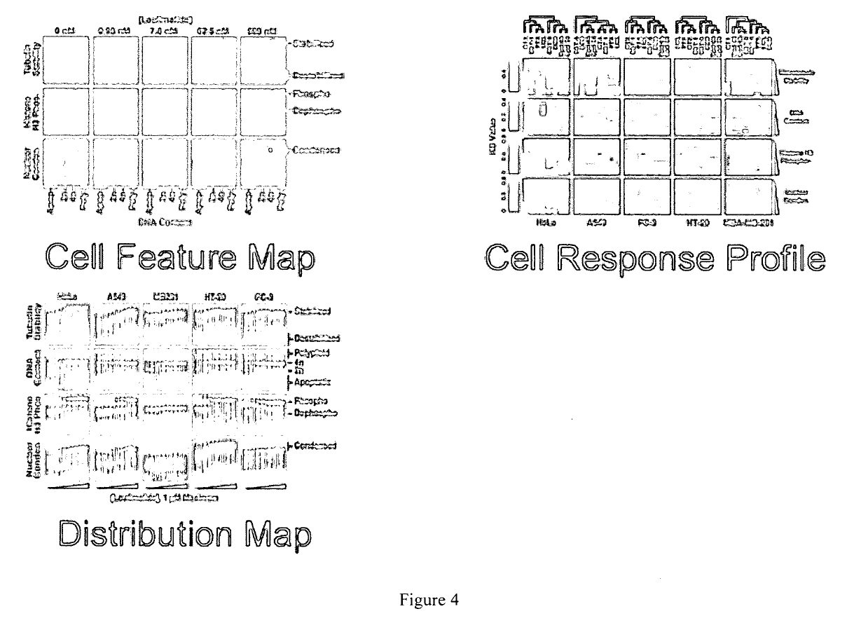

The practice of the inventive method also is aided through graphical analysis of cellular responses that contribute to a response profile. FIG. 4 illustrates some graphical display methods to display cellular responses that contribute to creating a cellular response profile. These graphical displays are also use to review multidimensional cellular responses. Cell feature maps (4A) are used to identify cellular functions that are associated with specific response profiles. Knowledge of the cell physiology events that lead to Apoptosis, as depicted here, can enhance the information in the output of a classifier, but is not necessarily required for the application of the method of this invention. Cell distribution maps (4B) depict the changes in the cellular response distributions, as the substance concentration is varied. These plots illustrate how the cells in a population can occupy discrete response classes, and move from class to class as substance concentration is varied. Cell response profiles (4C) are used to quantify the variation in population response distributions through the application of the KS analysis.

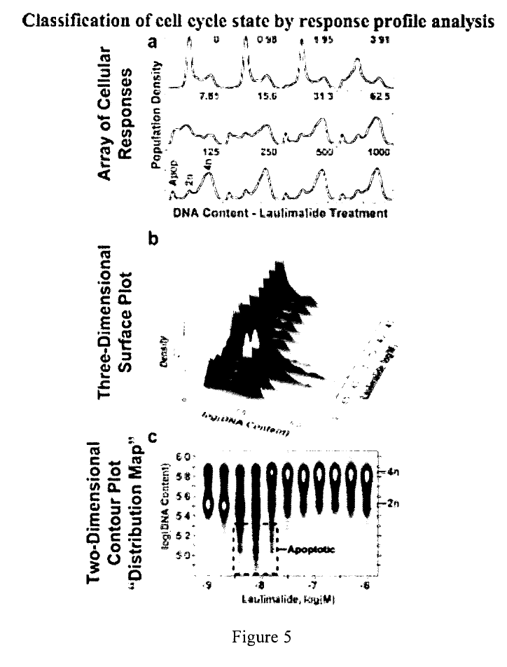

FIG. 5 depicts additional visualization tools used for cell population response profiles obtained from HCS analyses. A data set showing the effect of 11 concentrations of laulimalide (LML) on the DNA content of MDA-MB-23 1 breast tumor cells is presented using three visualization tools. FIG. 5a depicts an array of cellular response data plots. Each plot shows the population distribution of cellular DNA content at every concentration (nM) of LML. Subtle changes in the shapes of the population distributions were easily seen with this approach, but trends across the entire range of concentrations were difficult to discern. This is where KS analysis provides a more sensitive measure of the shift in an overall population response. 5b depicts a three-dimensional surface plot. When viewed at the optimal angle with the appropriate color encoding, a stacked series of cell population distribution curves provided an ideal context in which a series of complex curves could be simultaneously viewed and analyzed. However, comparisons between multiple three-dimensional surface plots on two-dimensional palettes such as computer screens or paper were problematic due to the awkward shape of the plots and lack of visual alignment cues. FIG. 5c depicts a two-dimensional contour plot or “Distribution Map” of the data. Color encoding of data point densities in Distribution Map can produce a unique approach for essentially projecting a three- dimensional surface plot onto a two-dimensional plane. For example, blue shades can encode the lowest population densities while shades of black and yellow can encode the highest population densities. Much of the detail provided by the three-dimensional surface plot was reproduced when the DNA content data were plotted as a Distribution Map. Furthermore, multiple Distribution Maps were easily arrayed for the simultaneous visualization of multiplexed HCS data sets.

In another embodiment, the invention provides a set of protocols and software tools used to carry out the profiling. Another embodiment of the invention is a panel of reagents and protocols for generating response profiles, either to create an knowledgebase, or to use with an existing knowledgebase and informatics software to profile substance physiological effects. Another embodiment of the invention is a database of physiological profiles. These could be provided as a product (i.e., a kit) to end users or used to perform profiling services for customers either with the inventive reagent panels and software or with the customer's own assays.

Accordingly, the invention provides a kit comprising reagents and instructions for using the reagents in accordance with the inventive method. In one embodiment, the kit comprises one or more reagents and instructions for employing the reagents to assay a battery of cells in accordance with a protocol involving incubating a battery of cells with a test or reference substance; acquiring images of cells within the battery; analyzing the images to measure or detect cellular features indicative of cellular functional classes; and creating a response profile comprising at least 6 of the cellular features. The kit can further include instructions for comparing the response profile of a test substance to a database of response profiles for substances with known biological systems effects. The reagents can include cells (e.g., preserved in liquid nitrogen), one or more fluorescent or luminescent labels, labware such as multiwell plates, culture medium, and the like. Furthermore, the kit can include a database of response profiles for substances with known biological systems effects (e.g., on electronic storage media). For example, the reagents specified in Table 7, 8 and 9 could be packaged in the appropriate amounts for the preparation of a standard number of assay plates, such as the 6 plates for processing the 16 compounds as described in Example 6. The kit would normally include a protocol for sample preparation, as described in Example 6, and optionally reference data values for compounds with know response profiles. This data could be provided in electronic format on an included CD or DVD disk or other data storage medium, as well as via network access to a centralized database of compound profiles. The selection, testing and validation of such reagent combinations and protocols requires significant effort to avoid interferences and ensure reliable performance, and therefore results in unique combinations of reagents and methods that are difficult to re-engineer, and enable multiplexed data acquisition used in profiling cellular activities.

The following examples further illustrate the invention but, of course, should not be construed as in any way limiting its scope.

### Example 1

This example demonstrates an embodiment of the invention in which a panel of assay function classes is used to profile substance toxicity.

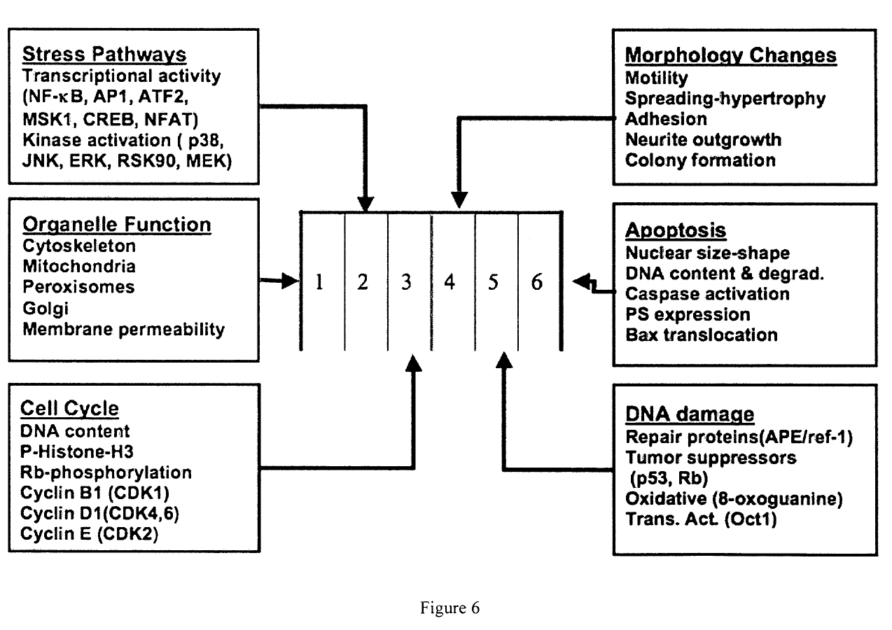

The function classes to be assayed for toxicity include Stress Pathways, Organelle Function, Cell Cycle Stage, Morphology Changes, Apoptosis and DNA Damage. Some features that can be assayed in accordance with the inventive method to produce a knowledgebase or to assay a test compound are presented in the following Table 1 and also in FIG. 6.

TABLE 1

| Cellular Function Class | Feature |
|---|---|
| DNA damage | i. Cell cycle regulation (DNA content and |
| | degradation) |
| | ii. Nuclear morphology |
| | iii. p53 protein activation |
| | iv. Rb protein phosphorylation |
| | v. Generation of 8-oxoguanine DNA damage |
| | product |
| | vi. Oct1 transcription factor activation |
| | vii. Activation of DNA repair proteins |
| | (APE/ref-1) |
| | viii. Histone H2A.X phosphorylation |
| Changes in phosphorylation | i. ERK |
| state of stress kinases | ii. JNK |
| | iii. p38 |
| | iv. RSK90 |
| | v. MEK |
| Apoptosis indicators | i. DNA content and degradation |
| | ii. Nuclear morphology |
| | iii. Caspase activation (multiple subtypes) |
| | iv. Mitochondrial function (mass-potential) |
| | v. Bax mitochondrial translocation |
| | vi. Cytochrome c mitochondrial release |
| | vii. PARP activation |
| Cell morphology and | i. Neurite outgrowth |
| differentiation | ii. Cell spreading and hypertrophy |
| | iii. Cell adhesion |
| | iv. Cell motility |
| | v. Colony formation-dispersal |
| Stress-induced transcription | i. NF-κB |
| factor activation or | ii. ATF-2 |
| inhibition | iii. CREB |
| | iv. AP-1 |
| | v. MSK |
| | vi. NFAT |
| | vii. Stat 1, 2, 3 |
| Metabolism | i. P-glycoprotein activity |
| | ii. CYP450 induction-inhibition |
| Cytoskeleton | i. Actin cytoskeleton stability |
| | ii. Microtubule cytoskeleton stability |

Within each of these assay function classes, one or more assays are selected to be used to measure one or more cellular features as an indication of a response in that assay function class. The methods of this invention can be used to validate additional assays and function classes which can be added a profile to improve the sensitivity, specificity or range of applicability of a specific embodiment of this invention.

One embodiment employs a panel of assays with one from each of these function classes. These assays are used first to build a predictive toxicology knowledgebase, and then to generate profiles of test compounds, to compare with the classes in the knowledgebase, and thereby to predict toxic affects of the test substances. Another embodiment of the invention uses all the assays listed in FIG. 6 to produce a more extensive profile, and then uses a statistical method such as principle components analysis to identify the features with the highest predictive power for a selected profile of toxicology parameters.

Reagents for assaying these cellular function classes and features are known to those of skill in the art and commercially available. Examples are presented in Table 2:

TABLE 2

| Cell Function | Cellular | | Reagent |
|---|---|---|---|
| Indications | Parameters | Reagent* | Source |
| Cell Proliferation | Cell Number | Hoechst 33342 | IVGN - H21492 |
| Cell Cycle | DNA Content | | Sigma - B2261 |
| Apoptosis | DNA Fragmentation | | AnaSpec - 83218 |
| Oxidative Stress | Increased Expression | Rb anti-HIF1α | Chemicon AB3883 |
| | Nuclear localization | Mo anti- HIF1α | Chemicon MAB5382 |
| | Nuclear Labeling | Mo anti-p-Hist. H2A.X | Upstate 05-636 |
| | | Mo anti-p-Hist H2A.X-FITC | Upstate 16-202A |
| | | Rb anti-p-Hist H2A.X | Chemicon AB3369 |
| | | Rb anti-p-Hist H2A.X | Upstate 07-164 |
| Stress Kinase Act. | Nuclear Labeling | Rb anti-p-c-Jun (ser 63) | Upstate 06-828 |
| | | Rb anti-p-c-Jun (ser 73) | Upstate 06-659 |
| | | Sh anti-p-c-Jun (T91/T93) | Upstate 07-570 |
| | | Sh anti-c-Jun | Chemicon CBL443 |
| Mitochondrial | Mitochondrial Intensity | Mitotracker Red CMXRos | Invitrogen M7512 |
| membrane | | Mitotracker Red CMH2XRos | Invitrogen M7513 |
| potential | | Mitotracker Orng CMTRos | lnvitrogen M7510 |
| | | Mitotracker Orng CMH2TRos | Invitrogen M7511 |
| | | Mitotracker Red 580 | Invitrogen M22425 |
| | | Mitrotracker Deep Red 633 | Invitrogen M22426 |
| DNA Damage | Nuclear Labeling | Mo anti-p53 | Chemicon CBL423 |
| | | Mo anti-p53 | Chemicon CBL422 |
| | | Rb anti-p-53 (ser 392) | Chemicon AB4060 |
| | | Mo anti-p53 (FITC) | Chemicon CBL423F |
| | Nuclear Labeling | FITC-streptavidin | Chemicon SA103 |
| | | Qdot 565-streptavidin | Chemicon SA302 |
| | | Qdot 655-streptavidin | Chemicon SA306 |
| Apoptosis | Mitochondrial Release | Sh anti-cytochrome c | Chemicon AB3547 |
| | | Mo anti-cytochrome c | Upstate 05-479 |
| | | Mo anti-cytochrome c | Chemicon MAB4612 |
| | Mitochondrial Release | Rb anti-AIF | Chemicon AB16501 |
| | | Rb anti-AIF | Chemicon AB16502 |
| Peroxisome Proliferation | Peroxisome Intensity | Mo anti-PMP70 | Affinity Bioreagents |
| | | | PA1-650 |
| | | Rb anti-PMP70 | Invitrogen 71-8300 |
| | | Rb anti-catalase | Chemicon AB1212 |
| Secondary Antibody | | Donkey anti-mouse Cy3 | Chemicon AP192C |
| Labels | | Donkey anti-mouse FITC | Chemicon AP192F |
| | | Donkey anti-mouse Cy5 | Chemicon AP192S |
| | | Donkey anti-rabbit Cy3 | Chemicon AP182C |
| | | Donkey anti-rabbit FITC | Chemicon AP182F |
| | | Donkey anti-rabbit Cy5 | Chemicon AP182S |
| | | Donkey anti-sheep Cy3 | Chemicon AP184C |
| | | Donkey anti-sheep FITC | Chemicon AP184F |
| | | Donkey anti-sheep Cy5 | Chemicon AP184S |

*All reagents must be validated for use in multiplexed immunofluorescence labeling of cells.

### Example 2

This example demonstrates a multiplexed HCS toxicity profiling panel.

This panel suitably is performed in assays of multiple cell types. All panels include cell cycle regulation (e.g., assayed by DNA content and degradation) as a function class and nuclear morphology measurements. Additionally, the following features that can be assayed in accordance with the inventive method to produce a knowledgebase or to assay a test compound are presented in the following Table 3:

TABLE 3

| Cellular Function Class | Feature |
|---|---|
| Apoptosis | 1. Mitochondrial mass |
| | 2. Mitochondrial cytochrome c release |
| | 3. Mitochondrial bax translocation |
| Cytoskeleton - stress kinase | 1. Actin cytoskeleton stability |
| | 2. Microtubule cytoskeleton stability |
| | 3. MAPK (ERK) activation |
| Neurotoxicity | 1. Neurite outgrowth |
| | 2. Microtubule cytoskeleton stability |
| | 3. Mitochondrial mass |
| | 4. Transcription factor activation (e.g., |
| | NF-κB, ATF-2, or other) |
| DNA damage response | 1. Histone H2A.X phosphorylation |
| | 2. p53 protein activation |
| | 3. Rb protein phosphorylation |
| Modulation of transcription | 1. Initiate activation with TNF-α and |
| factor activation | anisomycin mix |
| | 2. NF-κB or p38 activation |
| | 3. c-jun or ATF-2 activation |

### Example 3

This example demonstrates the use of RNAi knockdowns to provide additional systems cell biology information on the toxic response of cells.

Specific siRNA pretreatments can be overlayed into multiplex HCS toxicity profile panels, such as set forth in examples 1 and 2. Pretreatment of the cells with Cdc2 siRNA (Catalog #42819; Ambion, Inc.; Austin, Tex.) induces a G2 cell cycle block that can be exploited in a test for altered compound toxicity (e.g., by assaying for inhibition of apoptosis-inducing activity). Potential implementations of this strategy include (a) cross panels of siRNAs with multiplexed HCS assays in a single cell type and (b) cross sets of cell types with multiplexed HCS assays using a single siRNA pretreatment.

### Example 4

This example demonstrates the use of HCS toxicology profiling to extend toxicogenomics and whole animal studies.

Previously, toxicogenomics has been employed for predictive toxicology in drug development (see Carson et al., Cancer Res. 64:2096 (2004)). In this study, global changes in mRNA abundance in HeLa cells were measured after camptothecin treatment. Bioinformatics software was used to group the most significant camptothecin-regulated genes according to standardized gene ontology (GO) classifications. Various molecular pathways and cellular functions were identified as potential candidates for being involved in the toxic response: 1. p53-inducible genes (28.1% change), 2. Nuclear compartment genes (16.5% change), 3. NF-κB inducible genes (12.5% change), 4. Mitosis related genes (9.7% change), 5. Histone genes (8.1% change), and 6. Double strand DNA break repair genes (4.0% change). This study can be extended using a multiplexed HCS toxicology panel such as that set forth in Table 4:

TABLE 4

| Cellular Function Class | Feature |
|---|---|
| p53 activation and cell cycle | a. p53 protein activation |
| panel | b. Cell cycle regulation (DNA content |
| | and degradation) |
| | c. Retinoblastoma (Rb) protein |
| | phosphorylation |
| | d. RSK90 stress kinase phosphorylation |
| | e. Cdc2 siRNA pretreatment |
| Transcription factor panel | a. NF-κB activation-inhibition |
| | b. IRF-3 activation-inhibition |
| | c. Stat-3 activation-inhibition |
| Mitosis and histone modification | a. Histone H3 phosphorylation |
| panel | b. CENP-A phosphorylation |
| | c. Microtubule cytoskeleton stability |
| Histone and DNA double strand | a. Histone H2A.X phosphorylation |
| break-repair panel | b. ATM phosphorylation |
| | c. 8-oxoguanine generation |

### Example 5

This example demonstrates the use of HCS toxicology profiling using combined measurements of toxicity and potential for hepatic metabolism within a mixed population of cell types.

Liver-derived cells with specific drug metabolic activities are co-cultured with tumor-derived cells and the toxic responses of both cell populations are separately measured using multiplexed HCS toxicity profiling assays. The liver-derived cells with drug metabolism activities can, for example, be 1. Primary hepatocytes with constitutive mixes of CYP450 activities or 2. Liver-derived cells engineered to express specific CYP450 activities (e.g., 3A4, 1A2, etc.). Co-cultures of such liver-derived cells and tumor-derived drug target cells are generated in which the two populations are separately labeled such that the responses of the two populations can be separately measured. These co-cultures are then included in multiplexed HCS toxicity profiling assay panels such as described in other Examples.

The toxicity-metabolism screening system then can be validated using a set of drugs with known toxic effects, such as hepatitis, cholestasis, cirrhosis, jaundice, steatosis, and other hepatic metabolism potential. Moreover, the toxicity-metabolism system can be used to screen libraries of single compounds as well as combinations of compounds (e.g., drug-drug interactions).

### Example 6

This example pertains to a multiplexed toxicity HCS profiling panel. It describes the performance of a specific CellCipher™ cytotox profile which is designed to measure 11 cytotoxicity parameters using a two plate assay. The example also demonstrates how the resulting response data can be analyzed and interpreted.

Assay and Reagent Specifications. The Cytotox Profile Plate 1 contains the labels and features as indicated in Table 5, and the Cytotox Profile Plate 2 contains the labels and features as indicated in Table 6. The antibody and fluorescent indicators of cell physiology reagent specifications for Cytotox Profile Plate 1 are contained in Table 7 whereas the antibody and fluorescent indicators of cell physiology reagent specifications for Cytotox Profile Plate 2 are contained in Table 8. Finally, the assay buffer specifications for both Cytotox Profile Plates 1 and 2 are contained in Table 9.

TABLE 5
CellCipher ™ Cytotox Profile: Multiplex Plate 1

| Cell Parameter | Measurement | Reagents |
|---|---|---|
| (1) Cell Loss | % Cell Loss | Hoechst 33342 |
| (2) Cell Cycle Arrest | % Cells 2N | |
| (3) DNA Degradation | % Cells Sub 2N | |
| (4) Nuclear Size | Nuclear Area | |
| (5) Oxidative | Mean Nuclear | Mouse-anti-phospho- |
| Stress | Intensity Ch2 | histone H2A.X |
| | | FITC-donkey-anti- |
| | | mouse-IgG |
| (6) Stress Kinase | Mean Nuclear | Rabbit-anti-phospho-c-jun |
| Activation | Intensity Ch3 | Cy3-donkey-anti- |
| | | rabbit-IgG |
| (7) DNA Damage | Mean Nuclear | Sheep-anti-p53 |
| Response | Intensity Ch4 | Cy5-donkey-anti-sheep-IgG |

TABLE 6
CellCipher ™ Cytotox Profile: Multiplex Plate 2

| Cell Parameter | Measurement | Reagents |
|---|---|---|
| Cell Loss | Cell number | Hoechst 33342 |
| Cell Cycle Arrest | % Cells 2N | |
| DNA Degradation | % Cells Sub 2N | |
| Nuclear Size | Nuclear area | |
| (8) Mitochondrial | Mean Cytoplasmic | MitoTracker Red |
| Function I (potential) | Int. @ 30 min | |
| (9) Mitochondrial | Mean Cytoplasmic | MitoTracker Red |
| Function II (mass) | Int. 1-3 days | |
| (10) Mitosis marker | Mean Nuclear Int. | Rabbit-anti-phospho- |
| | | histone H3 |
| | | FITC-donkey-anti- |
| | | rabbit-IgG |
| (11) Microtubule | Mean Intensity | Mouse-anti-α-tubulin |
| Cytoskeleton Stability | over nucleus | Cy5-donkey-anti- |
| | | mouse-IgG |

TABLE 7
Reagent Requirements for Multiplex Plate 1.

| | | | Initial | Final Dilution | Exact volume of |
|---|---|---|---|---|---|
| | | | Dilution of | of Original | initial dilution |
| | | | Original | Reagent or | reagent required |
| Plate 1 Cell | | Catalog | Reagent | Final Reagent | for one 384-well |
| Parameter | Reagent | Number (lot) | (storage temp.) | Concentration | microplate |
| (1-4) Nucleus | Hoechst | Sigma B2261 | Reconstitute | 1:10000 | 0.38 μl |
| | 33342 | (044K4096) | with water to | | |
| | | | 10 mg/ml | | |
| | | | (4 C.) | | |
| (5) Oxidative | Anti- | Millipore | None. | 1:200 | 19.2 μl |
| Stress | phospho- | 05-636 | Already | | |
| Primary Ab | histone | (27505) | contains 30% | | |
| | H2A.X | | glycerol | | |
| | | | (−20 C.) | | |
| (5) Oxidative | FITC Anti- | Millipore | Reconstitute | 1:300 | 12.8 μl |
| Stress | mouse IgG | AP192F | with 400 μl of | | |
| Secondary Ab | | (0508006630) | 50% glycerol | | |
| | | | (−20 C.) | | |
| (6) Stress | Anti- | Millipore | None. | 1:200 | 19.2 μl |
| Kinase | phospho-c- | 06-659 | Already | | |
| Activation | jun | (28691) | contains 30% | | |
| Primary Ab | | | glycerol | | |
| | | | (−20 C.) | | |
| (6) Stress | Cy3 Anti- | Millipore | Reconstitute | 1:300 | 12.8 μl |
| Kinase | rabbit IgG | AP182C | with 400 μl of | | |
| Activation | | (0605029437) | 50% glycerol | | |
| Secondary Ab | | | (−20 C.) | | |
| (7) DNA | Anti-p53 | Calbiochem | Dilute with | 1:400 | 9.6 μl |
| Damage | | JA1308 | equal volume | | |
| Response | | (D252944) | of glycerol | | |
| Primary Ab | | | (−20 C.) | | |
| (7) DNA | Cy5 Anti- | Millipore | Reconstitute | 1:300 | 12.8 μl |
| Damage | sheep IgG | AP184S | with 400 μl of | | |
| Response | | (601021122) | 50% glycerol | | |
| Secondary Ab | | | (−20 C.) | | |

Reagent dead volumes are liquid handling equipment dependent. Typically, exact volume requirements must be increased by 10-20% to compensate for liquid handling equipment requirements.

TABLE 8
Reagent Requirements for Multiplex Plate 2.

| | | | Initial | Final Dilution | Exact volume of |
|---|---|---|---|---|---|
| | | | Dilution of | of Original | initial dilution |
| | | | Original | Reagent or | reagent required |
| Plate 2 Cell | | Catalog | Reagent | Final Reagent | for one 384-well |
| Parameter | Reagent | Number (lot) | (storage temp.) | Concentration | microplate |
| Nucleus | Hoechst | Sigma B2261 | Reconstitute | 1:10000 | 0.38 μl |
| | 33342 | (044K4096) | with water to | | |
| | | | 10 mg/ml | | |
| | | | (4 C.) | | |
| (8) | MitoTracker | Invitrogen | Reconstitute | 1:20000 | 0.19 μl |
| Mitochondrial | Red | M7512 | with DMSO | | |
| Function I | | (42746A) | to 1 mM | | |
| | | | (−20 C.) | | |
| (9) | Mito Tracker | Invitrogen | Reconstitute | 1:20000 | 0.19 μl |
| Mitochondrial | Red | M7512 | with DMSO | | |
| Function II | | (42746A) | to 1 mM | | |
| | | | (−20 C.) | | |
| (10) Mitosis | Anti- | Millipore | Dilute with | 1:400 | 9.6 μl |
| marker | phospho- | 06-570 | equal volume | | |
| Primary | histone H3 | (32219) | of glycerol | | |
| Antibody | | | (−20 C.) | | |
| (10) Mitosis | FITC Anti- | Millipore | Reconstitute | 1:300 | 12.8 μl |
| marker | rabbit IgG | AP182F | with 400 μl of | | |
| Secondary | | (508007651) | 50% glycerol | | |
| Antibody | | | (−20 C.) | | |
| (11) | Anti-α- | Millipore | Dilute with | 1:1000 | 3.8 μl |
| Microtubule | tubulin | 05-829 | equal volume | | |
| Cytoskeleton | | (32508) | of glycerol | | |
| Primary | | | (−20 C.) | | |
| Antibody | | | | | |
| (11) | Cy5 Anti- | Millipore | Reconstitute | 1:300 | 12.8 μl |
| Microtubule | mouse IgG | AP192S | with 400 μl of | | |
| Cytoskeleton | | (0604027318) | 50% glycerol | | |
| Secondary | | | (−20 C.) | | |
| Antibody | | | | | |

Reagent dead volumes are liquid handling equipment dependent. Typically, exact volume requirements must be increased by 10-20% to compensate for liquid handling equipment requirements.

TABLE 9
Assay Buffer Requirements for Multiplex Plates 1 and 2.

| | | | Exact volume of |
|---|---|---|---|
| | | | buffer required for |
| | | Catalog Number | one 384-well |
| Assay Step | Reagent | (lot) | microplate |
| Dilution of | Hanks Balanced Salt | Hyclone SH30030.03 | 15.4 ml |
| formaldehyde | Solution with Phenol | (AQL25083) | |
| | Red - 1x | | |
| Dilution of | Hanks Balanced Salt | Hyclone SH30030.03 | 6.1 ml |
| permeabilization | Solution with Phenol | (AQL25083) | |
| reagent | Red - 1x | | |
| Wash after fixation | Hanks Balanced Salt | Hyclone SH30268.02 | 38.4 ml |
| | Solution - 1x | (AQK24922) | |
| Wash after | Hanks Balanced Salt | Hyclone SH30268.02 | 38.4 ml |
| permeabilization | Solution - 1x | (AQK24922) | |
| Wash after primary | Hanks Balanced Salt | Hyclone SH30268.02 | 38.4 ml |
| antibody labeling | Solution - 1x | (AQK24922) | |
| First wash after | Hanks Balanced Salt | Hyclone SH30268.02 | 38.4 ml |
| secondary antibody | Solution - 1x | (AQK24922) | |
| labeling | | | |
| Second wash after | Hanks Balanced Salt | Hyclone SH30268.02 | 38.4 ml |
| secondary antibody | Solution - 1x | (AQK24922) | |
| labeling | | | |

Reagent dead volumes are liquid handling equipment dependent. Typically, exact volume requirements must be increased by 10-20% to compensate for liquid handling equipment requirements.

HepG2 cell handling and plating procedure. HepG2 cells were obtained from the American Type Cell Collection (cat no. HB-8065) and an original seed stock was prepared from one vial containing 1×10⁺⁶ cells. From the seed stock, a working stock was prepared using standard procedures. Cells were thawed form the working stock when required and maintained in culture for 20 passages before being discarded. Cells were maintained in MEM/EBSS (Hyclone SH30244.01) supplemented with 10% FBS (Hyclone SH30071.03), non-essential amino acids (Hyclone SH30238.01), penicillin-streptomycin-glutamine (Hyclone SV30082.01), and sodium pyruvate (Hyclone SH30239.01). Cells were maintained in T-150, vented, uncoated TC flasks (Coming 430825) using 20 ml culture medium. Cell passages were made approximately every 3-4 days when cells are approx 70% confluent and are made at 1:4 or 1:5 (approx 4×10⁺⁶ cells) using standard trypsinization methods.

Preparation of HepG2 cells for cytotox profile. The day prior to plating cells into microplates, HepG2 cells (70% confluent) were passaged by trypsinization, including trituration, and replated into the same flask from which they were removed.

Cell plating for cytotox profile. For the cytotox profile, thin bottom 384-well microplates were used that are compatible with the high numerical aperture optics available on most HCS readers. Falcon #3962 plates have the largest surface area and are suitable for HCS. These microplates were coated with collagen I coating, by rinsing the microplates with collagen I (Sigma C9791) solubilized in 1:1000 glacial acetic acid (Sigma A6283) at a concentration of 0.25 mg/ml and letting them air dry in a sterile hood produces a substrate for optimal attachment and spreading of HepG2 cells. The solubilized collagen I was added to dry 384-well microplates (16 μl/well), the plates were incubated at room temperature for 5 min, the solution was then shaken out of the wells, and the microplate left to air dry in a sterile hood. Cells were passaged by trypsinization, including trituration, and viable cells counted. Cell suspension (20 ml) was prepared per microplate at a concentration of 1.0, 2.0 or 3.5×10⁺⁶ cells/20 ml and 40 μl of cell suspension was plated into each well to yield the following cell densities for each time point: 30 min treatment-7000 cells per well; 24 h treatment-4000 cells per well; and 72 h treatment-2000 cells per well. After each microplate was filled, it was placed onto a stable benchtop to settle for 30 min. After 30 min settling at room temperature the microplates were placed into the 37 C 5% CO₂ incubator.

Compound preparation and treatment of cells. Standard compounds were prepared in DMSO (Sigma D8418) at the following concentrations: Camptothecin-Sigma C9911, 20 mM; Anisomycin-Sigma A9789, 10 mM; CCCP-Sigma C2759, 100 mM; and Paclitaxel-Sigma T7191, 5 mM. The test compounds were prepared in DMSO at concentrations up to 25 mM and stored at −20 C. All compound dilutions were performed in DMSO prior to further dilution in HBSS with phenol red. The maximal final concentrations of the standard compounds are as follows: Camptothecin-10 μM (200 μl of a 5× solution [50 μM] for each 3 plate set); Anisomycin-10 μM (200 μl of a 5× solution [50 μM] for each 3 plate set); CCCP-100 μM (200 μl of a 5× solution [500 μM] for each 3 plate set); and Paclitaxel-1 μM (200 μl of a 5× solution [5 μM] for each 3 plate set). A 10-point dilution set was made for each compound by diluting slightly more than 3-fold (square root of 10) on each step. Compound additions were made by transferring 10 μl of 5× compound stocks. For all conditions, DMSO was used at a final concentration of 0.4% in each well after compound addition (50 μl total volume).

Labeling of Cytotox Profiling Multiplex Plate 2 with MitoTracker Red before fixation. First, a 100 nM MitoTracker Red stock solution was prepared in warmed medium. To each well of the microplate, we added 50 μl of this 2× MitoTracker Red solution for a final concentration of 50 nM. The microplate was incubated for 5 min at 37 C in CO₂ incubator. The fluid was then removed from the microplate and 50 μl of cell culture medium was added to each well. The microplate was then incubated for 30 minutes at 37 C in CO₂ incubator before proceeding with the cell fixation protocol.

Cell fixation protocol. A 2× fixative was prepared containing formaldehyde (Sigma, 252549, 36% stock) at a concentration of 7.2% in HBSS with phenol red. To each well in the microplate, 50 μl fixative was added. The microplates were incubated for 30 min at room temp before being washed with HBSS (100 μl/well) which was immediately removed.

Cell permeabilization and labeling protocol. Cells were permeabilized by incubating with 0.5% (v/v) Triton X-100 (Sigma T9284) for 5 min at room temperature (16 μl/well). The microplates were washed with HBSS (100 μl/well) which was immediately removed. Cells in Multiplex Plate 1 were incubated with the primary antibody reagents as listed in Table 3 for 1 h at room temperature (10 μl/well). Cells in Multiplex Plate 2 were incubated with the primary antibody reagents as listed in Table 4 for 1 h at room temperature (10 μl/well). The microplates were washed with HBSS (100 μl/well) which was immediately removed. Cells in Multiplex Plate 1 were incubated with the secondary antibody reagents and Hoechst 33342 as listed in Table 3 for 1 h at room temperature (10 μl/well). Cells in Multiplex Plate 2 were incubated with Multiplex Plate 2 secondary antibody reagents and Hoechst 33342 as listed in Table 4 for 1 h at room temperature (10 μl/well). The microplates were washed twice with HBSS (100 μl/well) leaving the second wash in the wells. The plates were then sealed for HCS analysis.

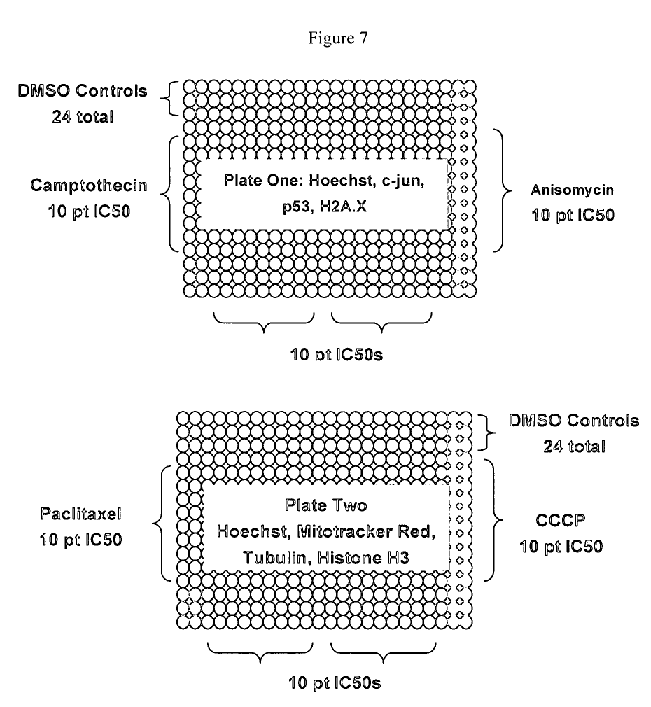

Standard plate layouts for CellCipher Cytotox Profiling Multiplex Plates. The standard plate layouts for Multiplex Plates 1 and 2 are depicted in FIG. 7. Each microplate contained 24 DMSO control wells distributed in the corners. Each microplate contained 2 duplicate standard toxin 10-point concentration series. Each microplate also contained 16 duplicate test toxin 10-point concentration series.

Reading plates. Cell imaging of prepared microplates or slides was performed with an ArrayScan® HCS Reader using the Cellomics® BioApplication Software coupled to a Cellomics® Store database. Other HCS readers and applications, as well as other microscope imaging systems, coupled with the same or alternative image analysis packages, can be used to perform data acquisition and feature extraction. Briefly, the instrument was used to scan one or more optical fields in each sample or microplate well, collecting four channels of fluorescence for each optical field on each plate.

Algorithms. The algorithms, encapsulated in the Cellomics BioApplication software produced multiple numerical feature values for each cell and for each well on each plate. Examples of cellular features include subcellular object total and mean intensities, shape features such as perimeter to area and length width ratio, and location for each cell within an optical field. Well features are averaged or accumulated over the whole population of cells measured in the well and include cell count, mean nuclear size, mean nuclear intensity, total nuclear intensity, mean cytoplasmic/nuclear ratio and along with the standard deviation of each of these mean values. Contingent on the effect that the added chemical compounds had on the attachment of cells to the substrate, the total number of cells measured per well was typically in the range of 100-1500, depending on the heterogeneity of the cellular response and the sensitivity of the assay. The assay output parameters were used to measure the 11 cytotox parameters shown in Tables 1 and 2 at 3 time points, acute (30 min), early (24 hour) and late (72 hour). For example, to calculate changes in nuclear morphology?, the average nuclear intensity value for each cell was used. The measurement of histone H3 phosphorylation was obtained using the average nuclear intensity of cells labeled with antibodies specific for phospho-histone H3. The specific image features used to extract information on the biological functions are listed in Tables 1 & 2. Those skilled in the art of imaging and cell analysis will recognize that there are many such algorithms readily available, and that there are many such cellular processes that are amenable to image-based analysis of cells to measure cellular functions.

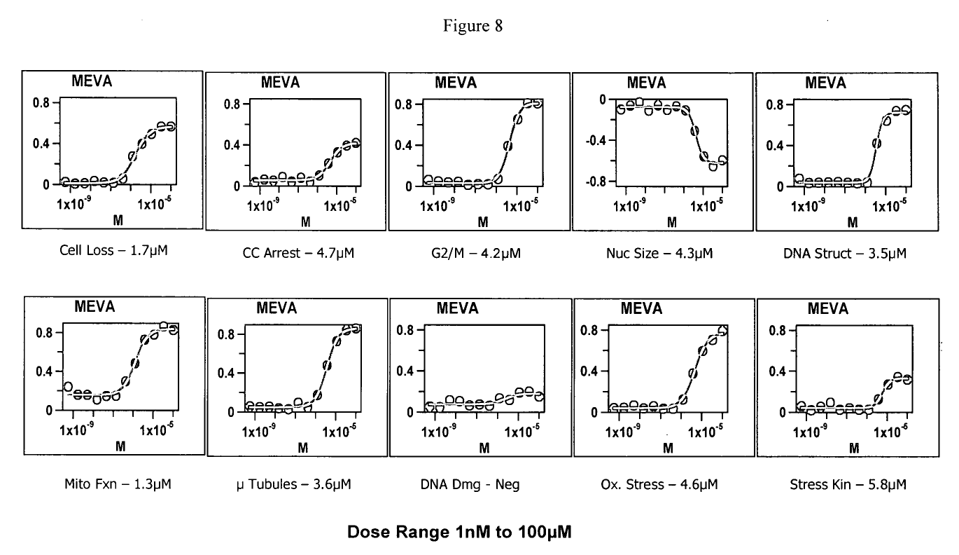

Quantifying the Response values To quantify overall changes in the cellular responses induced in a population of cells by treatment with reference or test molecules, the cellular response distribution for each cell parameter in a well was compared with that of control wells containing only DMSO using a non-parametric Kolmogorov-Smirnov (KS) goodness of fit analysis (KS value) (Giuliano et al., Assay Drug Dev Technol 2005; 3 (5):501-14).The KS analysis produced a single value for each well, and therefore, for each concentration. The dose-response data were fit to a 4 parameter logistics model using XL fit (IDBS, Guildford, UK). The IC50 values from the fits to the entire concentration series' were converted to a log scale (-log[IC50]). An example of the dose-response fits for a single time point for 1 compound, mevastatin, is illustrated in FIG. 8. The response values for all the compounds in this set were used to create a table.

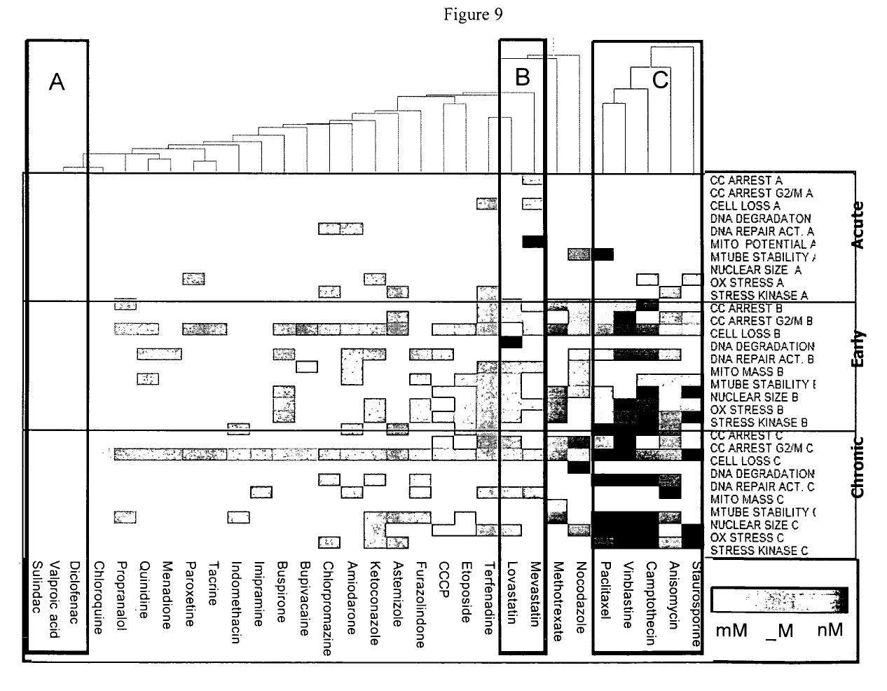

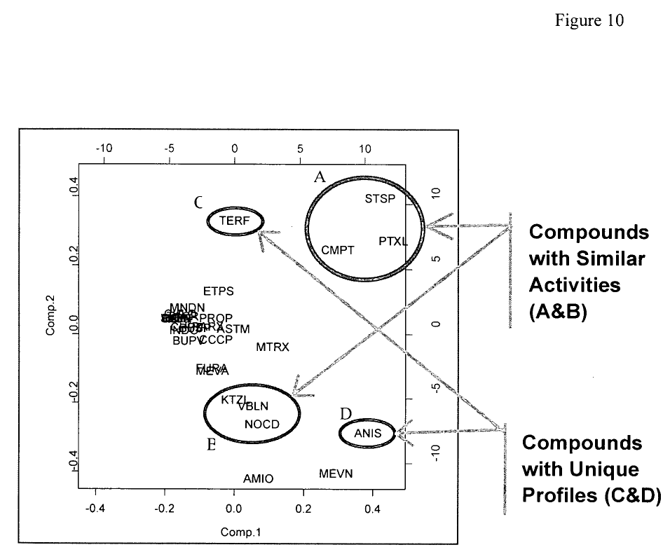

Clustering and Classification of Compound Responses. FIG. 9 is a heat map of the response values for all the compounds in this set. The compound names are along the horizontal axis and the measured features are plotted on the vertical axis. The measured features are in 3 groups; Acute are measured at 30 min, Early at 24 hours and Chronic after 72 hours of exposure. The gray level indicates the IC50 concentration, where white is mM and above, neutral gray is μM and black is nM and below. The compounds were clustered using a standard Euclidean distance metric. Those skilled in the art will recognize that many other metrics could also be used. The height of the dendrogram at the top indicates the degree of similarity between profiles, where shorter branches indicate that profiles are more similar. Three clusters of compounds are indicated by rectangles A-C. The 3 compounds in rectangle A have no activity in any of the assays, and thereby have a very high degree of similarity. The 2 compounds in cluster B, mevastatin and lovastatin have a moderate degree of activity (in the μM range) in many assays, have a very similar profile of activity across the assays, and in fact have very similar chemical structures. The 5 compounds in cluster C have a very high degree of activity (in the nM range) in many assays, and a varying degrees of similarity in their profiles. Even within this small data set, clustering on compound response profiles can be used to identify compounds that are chemically similar, as well as biologically similar. In addition to the many methods of cluster analysis, those skilled in the art of datamining will recognize that other statistical methods can be usefully applied to discover relationships in multidimensional data sets such as this. FIG. 10 illustrates a Principle Components (PC) plot of this same data set. Principle components analysis (PCA) is well known in the art and results in a linear mapping of the data into a set of orthogonal components that maximize the variance. FIG. 10 plots the first 2 PCs for the data in FIG. 9. The large cluster near the middle of the plot are compounds for which there is little or no discrimination in the first 2 PCs. However there are 2 significant clusters (A and B in FIG. 10) of compounds that are clearly discriminated from the rest, but similar to each other with respect to the first 2 PCs. There are also 2 compounds (C and D in FIG. 10), which are unique in this set with respect to the first 2 PCs. Several other compounds were also clearly discriminated in this plot. Analysis of the loadings of the first PC indicated that there was nearly equal contribution of many different assay features to the variance in the PC. The 10 most significant assays were: chronic oxidative stress, chromatin condensation, stress kinase activation, cell loss, DNA repair activity, nuclear size, and early stress kinase activation, oxidative stress, nuclear size, and cell loss. The PC loadings for these features ranged from 0.22-0.3 indicating that all contributed significantly to the discrimination of compounds by this profile. Analysis of loadings for other PCs indicates that even with this small library most of the assay features contributed significantly to discriminating the cellular modes of action for these compounds, The conclusion is that the breadth of assays in this profile provides an important tool for comparing compound activities and identifying common modes of action.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for predicting the biological systems effects of a test substance comprising:
a) providing a battery of cells to be treated;
b) incubating the cells with the test substance;
c) acquiring images of cells within the battery;
d) analyzing the images to measure or detect cellular features indicative of cellular functional classes;
e) creating a response profile comprising at least 6 of the cellular features; and
f) comparing the response profile of the test substance to a database of response profiles for substances with known biological systems effects; wherein the extent of correlation between the response profile of the test substance to the database of response profiles for substances with known biological systems effects indicates the probability that the test substance will exhibit a biological systems effect in a living cell, tissue or organism.

2. A method for constructing a database of response profiles for reference substances with known biological systems effects comprising:
a) providing a battery of cells to be treated;
b) incubating the cells with the a first reference substance;
c) acquiring images of cells within the battery;
d) analyzing the images to measure or detect cellular features indicative of cellular functional classes;
e) creating a response profile comprising at least 6 of the cellular features;
f) adding the response profile for the first reference substance to the database; and
g) optionally repeating steps a-f substituting a second reference substance for the first reference substance.

3. The method of claim 1 wherein, prior to acquiring the images, the cells contain one or more fluorescent or luminescent reporters.

4. The method of claim 3, wherein the cells express one or more fluorescent or luminescent reporters.

5. The method of claim 3, wherein one or more one or more fluorescent or luminescent reporters is introduced into the cells.

6. The method of claim 1, wherein the images of cells are obtained after labeling with one or more fluorescent or luminescent reporters targeting cellular features indicative of cellular functional classes.

7. The method of claim 3, wherein a reporter molecule is selected from the group consisting of fluorescent labels, fluorescent proteins, luminescent labels, and biosensors.

8. The method of claim 1, wherein, prior to acquiring the images, the cells contain one or more manipulation.

9. The method of claim 8, wherein a manipulation is selected from the group consisting of expression of a protein, knock-down of the expression of a protein, addition of a stimulus of known response or addition of a substance which induces differentiation of stem cells.

10. The method of claim 1, wherein, the cells are fixed prior to acquiring images of the cells.

11. The method of claim 1, wherein the cells are imaged live.

12. The method of claim 1, wherein images are analyzed using an algorithm to extract information from the images to produce outputs of the cellular features.

13. The method of claim 1, where the features are combined into a response profile using a method comprising cluster analysis.

14. The method of claim 1, wherein the cells are contacted with an array of substance concentrations and a response profile is constructed for each concentration.

15. The method of claim 1, wherein the battery of cells to be treated comprises 2 or more cell types.

16. The method of claim 1, wherein the scanning of the cells is repeated multiple times and analysis is performed at each time point to capture a kinetic response.

17. The method of claim 1, wherein the cellular features are selected from 2 or more functional response classes in the group consisting of cell proliferation, stress pathways, organelle function, cell cycle state, morphology, apoptosis, DNA damage, metabolism, signal transduction, cell differentiation and cell-cell interaction.

18. The method of claim 17 wherein the cellular features indicating cell proliferation are selected from the group consisting of nuclear count, cell count, total cell mass, total DNA, the phosphorylation state of cell cycle regulatory proteins, and the post-translational modification state of any protein involved in cell growth or division.

19. The method of claim 17 wherein the cellular features indicating stress pathway activation are selected from the group consisting of transcription factor activation of NF-κB, AP1, ATF2, MSK1, CREB, or NFAT, and kinase activation of p38, JNK, ERK, RSK90 or MEK.

20. The method of claim 17 wherein the cellular features indicating organelle function are selected from the group consisting of cytoskeletal organization, mitochondrial mass or membrane potential, peroxisome mass, golgi organization, and plasma membrane permeability.

21. The method of claim 17 wherein the cellular features indicating cell cycle state are selected from the group consisting of DNA content, Histone H3 phosphorylation state, Rb phosporylation state, cyclin B1 (CDKI) biosynthesis, cyclin DI (CDK4, 6) biosynthesis, and cyclin E (CDK2) biosynthesis.

22. The method of claim 17 wherein the cellular features indicating morphology are selected from the group consisting of motility, cell spreading, adhesion, ruffling, neurite outgrowth and colony formation.

23. The method of claim 17 wherein the cellular features indicating apoptosis are selected from the group consisting of nuclear size and shape, DNA content and degradation, caspase activation, phosphatidyl-expression, and Bax translocation.

24. The method of claim 17 wherein the cellular features indicating DNA damage are selected from the group consisting of repair protein (APE) expression, tumor suppressor (p53, Rb) expression, oxidative activity (8-oxoguanine), and transcription activity (Oct1).

25. The method of claim 17 wherein the cellular features indicating metabolism are selected from the group consisting of cAMP concentration, P-glycoprotein activity or CYP450 induction/inhibition, and the concentration of an added substance.

26. The method of claim 17 wherein the cellular features indicating signal transduction are selected from the group consisting of Ca++ ion concentration, (pH) expression of a protein, activation of a protein, modification of a protein, translocation of a protein, and interaction between proteins known to be associated with a specific pathway.

27. The method of claim 17 wherein the cellular features indicating cell differentiation are selected from the group consisting of expression of a tissue specific protein and exhibiting a tissue specific morphology.

28. The method of claim 17 wherein the cellular features indicating cell-cell interactions are selected from the group consisting of concentration of tight junction proteins at a cell-cell interface, and transfer of material from one cell to another.

29. The method of claim 1, wherein the cellular features are selected from 2 or more functional response classes in the group consisting of cell proliferation, cell cycle, apoptosis, oxidative stress, stress kinase activation, mitochondrial function, DNA damage, and peroxisome proliferation.

30. The method of claim 29, wherein one of the cellular features is cell loss.

31. The method of claim 29, wherein one of the cellular features is DNA degradation.

32. The method of claim 29, wherein one of the cellular features is cell cycle arrest.

33. The method of claim 29, wherein one of the cellular features is nuclear size.

34. The method of claim 29, wherein one of the cellular features is histone H2A.X phosphorylation level.

35. The method of claim 29, wherein one of the cellular features is c-jun phosphorylation level.

36. The method of claim 29, wherein one of the cellular features is p53 activation.

37. The method of claim 29, wherein one of the cellular features is mitochondrial membrane potential.

38. The method of claim 29, wherein one of the cellular features is mitochondrial mass.

39. The method of claim 29, wherein one of the cellular features is histone H3 phosphorylation.

40. The method of claim 29, wherein one of the cellular features is microtubule stability.

41. The method of claim 1, wherein profiles are built from the feature measurements comprising:
a) calculating a parameter such as Kolmogorov-Smirnov values or average values as a measure of cell population shifts for each feature measurement at each compound concentration for each compound to generate parameters for dilution series,
b) fitting such dilution series parameters using a 4-parameter logistic fit;
c) analyzing the resulting fitted data to calculate EC50 values;
d) converting the EC50 values to log scale as a measure of compound activity; and
e) using cluster analysis to identify similarities in profiles as well as correlations between cellular systems responses.

42. A kit comprising one or more reagents and instructions for employing the reagents to assay a battery of cells in accordance with a protocol involving
a) incubating a battery of cells with a test or reference substance;
b) acquiring images of cells within the battery;
c) analyzing the images to measure or detect cellular features
d) indicative of cellular functional classes; and
e) creating a response profile comprising at least 6 of the cellular features.

43. The kit of claim 42, further comprising instructions for comparing the response profile of a test substance to a database of response profiles for substances with known biological systems effects.

44. The kit of claim 42, further comprising instructions for adding the response profile of a reference substance to a database of response profiles for substances with known biological systems effects.

45. The kit of claim 42, further comprising a database of response profiles for substances with known biological systems effects.

46. The kit of claim 42, wherein one or more reagents comprise a fluorescent or luminescent label.

47. The kit of claim 42, wherein one or more reagents comprise a culture of cells.

48. A database constructed in accordance with the method of claim 2.
